(19) United States
(12) Patent Application Publication
(10) Pub. No.: US 2005/0149071 A1
(43) Pub. Date: Jul. 7, 2005

(54) Anastomosis device, tools and method of using

(76) Inventors: Ryan Abbott, San Jose, CA (US); Greg C. Liu, Sunnyvale, CA (US)

Correspondence Address: LAW OFFICE OF ALAN W. CANNON, 834 SOUTH WOLFE ROAD, SUNNYVALE, CA, 94086, US

(21) Appl. No.: 10/746,966

(22) Filed: Dec. 24, 2003

(51) Int. Cl. A61B 17/08

(52) U.S. Cl. 606/153

(57) ABSTRACT

Anastomosis devices, tools and methods of performing sutureless anastomosis.

### FIELD OF THE INVENTION

The present invention relates to the field of surgery. More particularly, the present invention relates to devices, tools and methods for performing sutureless anastomoses.

### BACKGROUND OF THE INVENTION

There are many medical procedures which require the performance of one or more anastomoses in which a conduit such as a vessel, duct, graft or other tubular structure must be joined to another vessel, duct, or other hollow structure such as an organ to establish continuity between these structures. One of the more prevalent needs for improve anastomosis techniques lies with the treatment of coronary artery disease, where a stenosis of one or more coronary arteries prevents or seriously interferes with a normal blood supply to the heart tissue. In such situations, a total or partial blockage of a coronary artery is often treated by bypassing the obstruction in a heart bypass procedure, such as a coronary artery bypass graft (CABG) procedure, in which a graft is fluidly connected to the blood supply on opposite sides of the site of the stenosis to provide an alternate route for the blood to take on route to the heart.

The graft may be natural conduit, artificial conduit, or a combination of natural and artificial conduits. Typically, a natural conduit in the form of an autograft harvested from the patient is used. Common natural conduits include the saphenous vein from the leg, the radial artery from the arm, or the internal mammary artery rerouted to be anastomosed downstream of the site of the stenosis.

Conventional CABG procedures are currently performed while the beating of the heart has been stopped, with the circulation and oxygenation of the patient's blood being performed by a heart and lung bypass machine. This procedure requires significant manipulation and clamping of the aorta of the patient. Recently, it has been found that this procedure tends to increase the risk of dislodging plaque that may have accumulated on the internal wall of the aorta in the vicinity of the clamping. Dislodgment of plaque can cause emboli in various locations in the patient's body, cutting off the blood supply downstream of the locus of the embolus, which can cause a stroke or other serious medical complications. Further, the heart-lung bypass machine is thought to cause mechanical damage to the blood cells which furthers the risk of medical complications, due to potential clot formation.

Recently there has been an increase in the performance of beating heart CABG procedures, in which the bypass of one or more stenoses is performed while the patient's heart continues to beat, with the circulation and oxygenation of the patient's blood being performed naturally by the heart and lungs of the patient. While beating heart procedures reduce the associated risks of stroke and other post-operative complications associated with the clamping and manipulation of the aorta and the use of the heart-lung bypass machine, they also tend to increase the difficulty somewhat in performing what were already difficult and delicate anastomosis procedures that must be performed to connect the bypass graft or grafts during the CABG procedure.

The most conventional techniques for making anastomoses involves manually suturing the two tubular conduits together (e.g., manually suturing the graft to the target vessel) around an opening between them. Manual suturing is difficult, time-consuming and requires a great deal of skill and manual dexterity on the part of the surgeon performing the anastomosis. The difficulties in performing anastomoses by manual suturing are magnified when they are done during a beating heart CABG procedure as the beating of the heart introduces perturbations that make it even more difficult to suture in a reliable, consistent and efficient manner. These difficulties have largely limited CABG procedures to open surgical settings which provide sufficient surgical access and visualization to complete the delicate anastomoses.

Thus, there is a need for sutureless anastomosis devices, tools and techniques that offer a reliable alternative to suturing techniques, and which are relatively easier to implement while giving consistent results. It would further be desirable to provide such devices, tool and techniques that would facilitate the performance of higher quality anastomoses than those currently made and with less time required to make the anastomoses.

With continued interest and development toward CABG procedures which are even less invasive than the current techniques for beating heart CABG procedures, it will further be desirable to provide anastomosis techniques which can be performed endoscopically, with the surgeon working outside of the patient.

### SUMMARY OF THE INVENTION

Devices for use in making an anastomosis between tubular fluid conduits in the body of a patient are described. The anastomotic device includes a unitary structure having a main body disposed annularly about a longitudinal axis and having first and second end portions; a plurality of members extending radially outwardly from the first end portion; and the second end portion having a plurality of spaced struts adapted to buckle in a radially outward direction upon axial compression of the device.

The device may further include a second set of spaced struts which are collapsible secondarily to the first set of struts, and over a variable range of distance to accommodate for varying wall thicknesses of the tubular conduits being joined by anastomosis.

The struts may be joined by a set of ring members to define the annular shape of the main body. A proximal end of the device includes members extending radially outwardly from the first end portion of the device. The radially extending members may extend from a proximal ring member. Where two sets of struts are provided, a third ring member may be provided to join the first and second sets of struts.

Graft retaining members, such as tines may extend radially outwardly from the second end portion of the device. Upon loading a graft on the device, the graft is passed through an internal annular space defined by the main body of the device, and then everted over the second end of the device to be retained by the graft retaining members.

One or more locking members, such as locking tines, may be provided integrally with the second end portion of the device and slidably connecting with the first end portion. Upon buckling the struts of the device, the locking member or members slide with respect to the first end portion and extend beyond the first end portion. The locking member or member can then be bent over the first end portion to lock the relative positions of the first and second end portions in the buckled configuration.

The first end portion may include a plurality of eyelets axially aligned with the locking members for slidably receiving free ends of the locking members.

The struts of the second end portion of the device, upon buckling, are adapted to form a compression fit with the members extending radially outwardly from the first end portion to form a seal between the everted end of the graft vessel and an inner wall of a target vessel.

The distal end portion of the device may be adapted to further evert the conduit or graft retained thereon, upon buckling.

A deployment instrument for deploying an anastomosis device according to the present invention is provided to capture an anastomosis device adapted for making an anastomosis between tubular fluid conduits in the body of a patient and comprising a unitary structure having a main body disposed annularly about a longitudinal axis, having first and second end portions and configured to be loaded with a first of the two conduits to be joined by the anastomosis, wherein the conduit is loaded by passing a free end thereof through an internal space defined by the main body in a direction from the first end portion to the second end portion and everting an end of the first conduit over the second end portion. The deployment instrument includes first and second tubes concentrically arranged for axial sliding movement with respect to one another.

The first tube has a first outside diameter and further has a gradually increasing second outside diameter on a distal end portion thereof. The second tube has an inside diameter slightly greater than the first outside diameter of the first tube so that the second tube is free to slide with respect to first tube along the portions defined by the first outside diameter. The second tube further has radially expandable members defining a radially expandable distal end portion. Upon sliding the radially expandable distal end portion into contact with distal end portion of the first tube, or upon sliding the distal end portion of the first tube into contact with the radially expandable distal end portion, the gradually increasing outside diameter of the first tube distal end portion drives the radially expandable members radially outward to assume an expanded conformation. Upon sliding the distal end portion of the first tube out of contact with the radially expandable distal end portion or vice versa, the radially expandable members return to an unbiased, non-expanded configuration.

The first and second tubes of the deployment instrument are each provided with a longitudinal slot. The longitudinal slots align with one another and are configured to allow the first conduit or graft to pass therethrough. This feature allows side loading of the deployment device so that a graft or other conduit loaded on an anastomosis device need not have a second free end to be loaded into the deployment device.

The first and second tubes are configured to slide through the internal space defined by the main body of the device, in a direction from the first end portion to the second end portion, between an external wall of the first conduit or graft and an internal wall of the device, when the radially expandable members are in the unbiased, non-expanded configuration.

The first and second tubes can then be used to capture the device after sliding through the internal space. The capture is effected upon expanding the radially expandable members by moving the distal end portion of the first tube into contact with the radially expandable members. The radially expandable members, upon radially expanding, may contact and exert a force against the internal wall of the device. The distal ends of the radially expandable members may be provided with catch members that abut a distal end of the device upon radial expansion of the radially expandable members to capture the device.

The deployment instrument is further adapted to buckle the device for joining the first and second conduits. A stop member may be provided proximally of the distal end portions of the tubes. The first end portion of the device abuts against the stop member upon capture of the device. The first and second tubes are axially slidable in a proximal direction with respect to the stop member to exert a compressive force on the device to buckle it.

The first and second tubes are axially slid in a distal direction to release the compressive force after completion of the buckling of the device. The first tube is then slid still further distally with respect to the second tube, in order to take the distal end portion of the first tube out of contact with the radially expandable members. The radially expandable members accordingly return to the non-expanded configuration so that the buckled device may be slid off the distal ends of the first and second tubes.

The deployment instrument may be further provided with a third tube having an inside diameter slightly greater than an outside diameter of the second tube. The third tube may be linked with the first tube, so that when the first tube is axially slid within the second tube, the third tube axially slides over the outside of the second tube along with the sliding of the first tube. The third tube has an outside radius greater than a radial extent of the catch members of the second tube when they are in the non-expanded configuration. In this way, the third tube prevents the buckle device from catching on the catch members as it is released from the deployment instrument.

The deployment instrument may be further adapted to lock the device after buckling the device, with the provision of a device lock. The device may be provided with at least one locking member which slides past a proximal end of the device upon compression of the device and which is connected with a distal end of the device. After buckling the device, the device lock of the instrument is slid distally with respect to the first and second tubes, wherein it abuts the at least one extending locking member and bends it over against the proximal end portion of the device, thereby locking the relative positions of the first and second end portions of the device.

A force limiter may be provided in the deployment device to interconnect the second tube with a relatively fixed portion of the instrument. The force limiter limits an amount of compressive force that the second tube can apply to the device during buckling.

A method of performing an anastomosis to join a first conduit to a second conduit is described to include: inserting a free end of the first conduit through an annular spaced defined by an anastomosis device comprising a unitary structure having a main body disposed annularly about a longitudinal axis and having first and second end portions; at least one first end member extending further radially outward than a radial extent of the annularly disposed main body; and graft retaining members extending from the second end portion, the graft being inserted in a direction from the first end portion to the second end portion so that the free end extends from a second end of the device; everting the extending free end of the graft over the second end of the device and retaining the everted free end with the graft retaining members; forming an opening through a wall of the second conduit, wherein the opening is dimensioned to allow the everted end and main body, but not the at least one first end member to pass therethrough; inserting the device and graft into the opening until the at least one first end member abuts the external wall of the second conduit; and compressing the device to buckle the second end portion, wherein the second end portion, upon buckling is no longer capable of passing back through the opening.

The compressing is performed only up until a pre-defined compression force has been reached. The compressing may further at least partially collapse the first end portion after buckling the second end portion.

The method may further include locking the relative positions of the first and second end portions after completion of compression.

A method of preparing a graft vessel and performing an anastomosis to join the graft to a target vessel is described to include: measuring an outside diameter and wall thickness of the graft vessel; selecting an appropriately sized anastomosis device, based on the outside diameter and wall thickness measurements; loading the graft vessel on the anastomosis device so that the graft vessel passes through a longitudinally extending annular space defined by a main body of the anastomosis device, extends beyond a distal end of the anastomosis device and is everted back over an external surface of the distal end of the anastomosis device; selecting a punch appropriately size matched to the outside diameter and wall thickness measurements and punching an opening through a wall of the target vessel; inserting the loaded graft into the opening, wherein the anastomosis device has an enlarged proximal end that is incapable of passing through the opening and abuts against the wall of the target vessel upon inserting the loaded graft; and buckling the anastomosis device so that a distal end portion thereof increases in diameter and compresses the everted end of the graft vessel against an internal wall surface of the target vessel.

The method of anastomosis may be performed either with a graft having two free ends or with a graft having only one free end.

These and other objects, advantages, and features of the invention will become apparent to those persons skilled in the art upon reading the details of the devices, tools and methods as more fully described below.

### BRIEF DESCRIPTION OF THE DRAWINGS

FIG. 1A shows a flat pattern of an anastomosis device, according to the present invention.

FIG. 1B shows a flat pattern of another anastomosis device, according to the present invention.

FIG. 2 is a three-dimensional, perspective view of the device shown in FIG. 1B.

FIG. 3 shows a flat pattern of another example of an anastomosis device, according to the present invention.

FIG. 4 is a perspective view of a deployment instrument according to the present invention.

FIG. 5 is an enlarged view of the distal tip portion of the instrument shown in FIG. 4.

FIG. 6 shows a graft having been loaded on a device and captured by a deployment instrument according to the present invention.

FIG. 7A is a partial top view showing the gradually increasing diameter of the distal end portion of the wedge tube of the deployment instrument.

FIG. 7B is an end view of the expandable catch cam members at the distal end portion of the catch cam tube.

FIG. 7C shows the interaction between the distal end portion of the wedge tube the catch cam members of the catch cam tube during a capture procedure for fixing an anastomosis device on the deployment tool.

FIG. 7D shows an anastomosis device 1 having been captured on a deployment tool according to the present invention.

FIG. 8A shows a partial view of a proximal end portion of a deployment tool according to the present invention.

FIG. 8B shows an exposed view of working components in the mechanism for operating a deployment tool according to the present invention.

FIG. 8C shows a partial assembly of a deployment tool according to the present invention.

FIG. 8D shows another exposed view of working components in the mechanism for operating a deployment tool according to the present invention.

FIG. 9A is a perspective view of a graft vessel having been passed through the annular space defined by an anastomosis device according to the present invention.

FIG. 9B is a perspective view of the graft vessel shown in FIG. 9A after further having been everted and pierced by graft tines.

FIG. 10A is a perspective view of a graft vessel having been passed through the annular space defined by an anastomosis device having shortened or no tines according to the present invention.

FIG. 10B is a perspective view of the graft vessel shown in FIG. 10A after further having been everted over the device.

FIG. 11 is a view of the graft and anastomosis device of FIG. 9B, after bending over the graft tines to further secure the graft to the device.

FIG. 12 is a partial perspective view showing the opening in the target vessel into which the graft and anastomosis device are to be inserted.

FIG. 13 is a schematic partial view showing insertion of a graft and anastomosis device into an opening in a target vessel using a deployment device according to the present invention.

FIG. 14 is a schematic view showing insertion of a graft and anastomosis device into an opening in a target vessel using a deployment device according to the present invention.

FIG. 15A is a sectional schematic view of a graft and anastomosis device having been inserted into a target vessel.

FIG. 15B is a sectional schematic view of the graft and anastomosis device of FIG. 15A after buckling the distal end portion of the anastomosis device.

FIG. 15C is a sectional schematic view of the graft and anastomosis device shown in FIG. 15B after having partially collapsed the proximal end section and after beginning to lock the locking tines.

FIG. 15D is a sectional schematic view of the graft and anastomosis device shown in FIG. 15C after having locked the locking tines.

FIG. 16 is a top view of a completed anastomosis viewed on the inside wall of a target vessel.

FIG. 17 is a perspective view of an aortotomy punch usable for forming an opening in a target tubular member, for forming an anastomosis at the site of the opening.

### DETAILED DESCRIPTION OF THE INVENTION

Before the present devices, tools and methods are described, it is to be understood that this invention is not limited to a particular device, method step or tool described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a tine” includes a plurality of such tines and reference to “the strut” includes reference to one or more struts and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “tine” is used herein to denote an elongated structure forming a portion of an anastomosis device as described. A “tine” generally has a free end which can have any of a variety of tip configurations, including either a pointed or non-pointed tip.

A “strut” is defined herein to refer to a structural supporting or connecting element which joins at least two other components of an anastomosis device, such as two rings, for example.

A “ring” as used herein, refers to a body-shaping member of the anastomosis device which forms a general configuration over which a graft is mounted.

The present invention provides devices, tools and methods for joining two tubular conduits, such as vessels, organs or other tubular formations, particularly for forming anastomoses in cardiovascular applications, such as those required during the performance of a cardiopulmonary bypass. The present invention avoids the need by prior anastomosis techniques wherein the aorta is clamped to interrupt blood flow to the area of the aortic wall to which a vein or other conduit is to be anastomosed. Such clamping may result in liberation of plaques and tissue fragments which can lead to organ dysfunction, such as strokes, renal failure, or intestinal ischemia. The anastomosis techniques according to the present invention do not require any significant additional space surrounding the site of the anastomosis and inside the patient to connect the anastomotic device to the target vessel. According to the invention, a sutureless connection can be provided between a graft and a target vessel, while minimizing thrombosis or restenosis associated with the anastomosis. The devices allow the anastomosis to be performed very rapidly, with high reproducibility and reliability, without clamping, and with or without the use of cardiopulmonary bypass.

Device

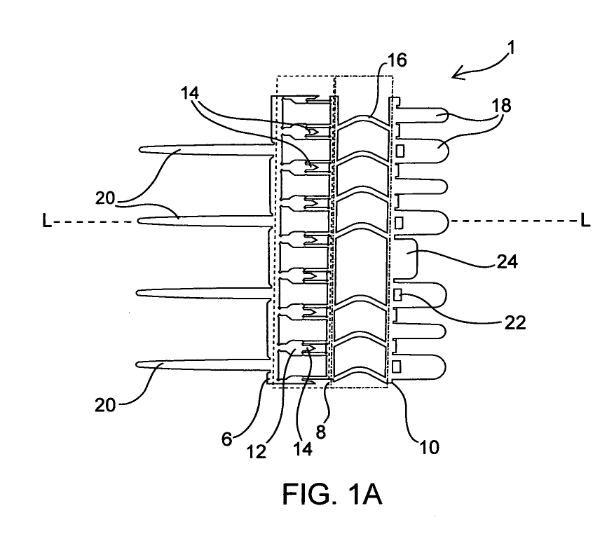

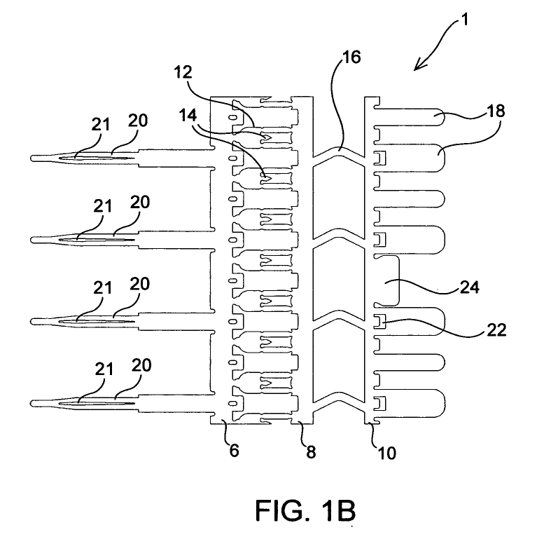

FIG. 1A shows, for ease of description, a flat pattern of an anastomosis device 1 according to the present invention. Practically speaking, device 1 is generally formed integrally, as an annular structure, such as by laser cutting from tubular stock, for example, although it would be possible to cut or stamp a planar structure from a sheet of material and then weld or otherwise fix the device in its annular form. FIG. 1A therefore shows the device 1 as if it were cut along a line parallel to its longitudinal axis L and then flattened into a planar form. The device 1 may be made from any suitable medical grade material, including stainless steel, or from other materials having appropriate performance characteristics, such as tantalum, tungsten or platinum, for example. Preferably, device 1 is made from a plastically deformable material such as 316L stainless steel.

The device 1 can be formed in various sizes to suit the dimensions of a graft or vessel to be joined to another site. For purposes of establishing a proximal anastomosis during performance of a coronary bypass procedure, devices 1 having outside diameters 2 varying within the range of about 3.0 mm to about 7.0 mm, a material thickness of about 0.007″±0.003″, and having an initial length 4 of about 0.2″ to about 0.7″, generally about 0.25″, so that they are adapted to accommodate anastomosis of a graft to aortas having wall thicknesses within the range of about 1 mm to about 5 mm.

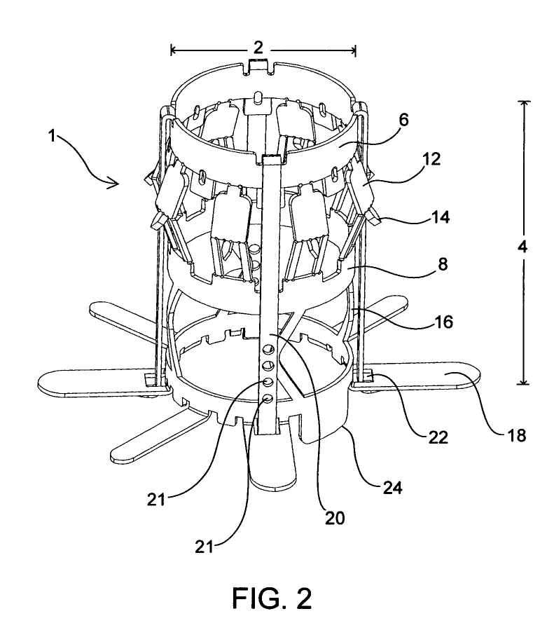

Device 1 includes three rings 6, 8 and 10 which form a framework of a generally cylindrical structure as can be seen in FIG. 2. Buckling struts 12 join rings 6 and 8 and are generally equally spaced around the circumferences of the rings 6 and 8 to form a buckling portion of the device 1. Buckling struts 12 are bent outwardly from an outer surface of an imaginary cylinder defined by rings 6, 8 and 10, to make the buckling portion more susceptible to collapse than the remainder of device 1 upon exertion of compressive forces along the longitudinal axis of device 1. Buckling struts 12 are further cut out to form graft tines 14, which further weaken the buckling struts to make them more susceptible to buckling. Graft tines 14 are bent to positions substantially perpendicular to the longitudinal axis of device 1 during forming, to position them for anchoring the end of a graft, which function is discussed in greater detail below. Alternatives to graft tines include spikes, glue, a rubber pad that is stegging, or other features designed to hold the graft in an everted configuration during performance of an anastomosis. Another alternative is to completely forego tines or any other structure for holding the graft in the everted configuration, and instead, to simply evert the graft end over the structure of the device 1.

Support struts 16 join rings 8 and 10 and are generally equally spaced around the circumferences of the rings 8 and 10 to form a supporting portion of the device 1, which buckles only secondarily to the buckling portion. Support struts 16 are angled to enhance their buckling, but, in contrast to buckling struts 12, the bending angle of the support struts 16 is such that support struts 16 maintain conformity with the imaginary cylindrical surface defined by rings 8 and 10. Comparatively, when the buckling section collapses, buckling struts bend outwardly so as to effectively increase the outside diameter of that portion of the device 12, while, in contrast, struts 16 tend to bend or buckle in a direction substantially perpendicular to the direction that struts 12 bend in, so that the struts 16, even after bending, substantially conform to the imaginary cylindrical surface and do not substantially increase the outside diameter of the support portion of the device 1.

External tines 18 extend from ring 10 and are bent substantially perpendicularly to the longitudinal axis L of device 1 during forming. External tines 18 form the contact surface by which device 1 applies pressure to the external surface of a vessel (e.g., external wall of the aorta) to which a graft held by device 1 is being joined. Locking tines 20 extend from ring 6 at substantially evenly spaced locations about the circumference of ring 6. Locking tines 20 have a sufficient length to span the remaining length of device 1 when they are folded over by one hundred and eighty degrees during forming. The external tines 18 which are aligned with locking tines 20 contain locking receptacles 22 through which the respective locking tines 20 pass upon folding them back one hundred and eighty degrees during forming. The locking tines 20 are bent over to the external side of the general cylindrical shape of device 1, and threaded through the locking receptacles 22 on the external tines which extend radially away from the general cylindrical shape of the device 1, as shown in FIG. 2. By passing locking tines 20 through receptacles 22, locking tines 20 effectively link rings 6 and 10 to provide an important locking feature upon deployment of the device, as will be discussed below. The external tines 18 that contain the locking receptacles 22 may be formed wider than the external tines 18 that do not contain locking receptacles, to compensate for the loss of surface area due to formation of the locking receptacle, as well as to provide a greater surface area against which the respective locking tines 20 are forced.

FIG. 1B shows another example of a flat pattern of an anastomosis device 1 according to the present invention, before any forming of the device has been performed. As is the case with the device 1 in FIG. 1A, device 1 (FIG. 1B) is generally formed integrally, as an annular structure (e.g., see FIG. 2), such as by laser cutting from tubular stock, for example, although it would be possible to cut or stamp a planar structure from a sheet of material and then weld or otherwise fix the device in its annular form. The device 1 in FIG. 1B is substantially similar to that of the device of FIG. 1A, and therefore all of the description of the features will not be repeated here, but a focus on the main differences between the devices will be described. It will be readily apparent that a fewer number of support struts 16 are provided in the device of FIG. 1B. By providing a fewer number of support struts 16 it is believed that a tendency of the struts 16 to buckle outwardly or inwardly is greatly reduced. In any of the designs described herein, a deployment tool (described below) tends to prevent buckling inwardly, but with the currently described design, the locking tines 20 are much more effective in preventing outward buckling of the support struts 16.

Another significant difference in the device of FIG. 1B is that the graft tines 14 formed in buckling struts 12 are formed to have a shorter length than those in the device of FIG. 1A. For example, graft tines 14 in the embodiment of FIG. 1B are generally formed to have a length less than about 0.25 mm so that it will be impossible to pierce the entire wall of the graft and extend out the everted side of the graft wall. The shorter graft tines 14 cannot extend all the way through the wall of the graft when it is mounted thereon, and, accordingly, the graft tines 14 do not extend from the everted wall of the graft when mounted. When the device 1 and graft 3 are deployed to form the anastomosis, the metal tines are not exposed in the completed anastomosis, as will be shown and described as the description proceeds. The graft tines 14 of the device in FIG. 1A, on the other hand, are of a length which can and often do extend through the wall of the graft.

Locking tines 20 include weakened sections or cutouts 21 which assist in the preferential bending of the tines in the locations of the weakened sections during the locking phase of deployment of the device. This helps ensure that the locking tines bend into the configuration for which they have been designed, thereby providing the intended secure locking function. Weakened section 21 can be formed by elongated slots, as shown in FIG. 1B, or a series of holes, as shown in FIG. 2, or other shapes and configurations of cutouts designed to weaken the intended sections of the tines where it is desired to have the bending of the tines begin during the locking phase.

FIG. 2 shows device 1 in its three dimensional configuration, which may be formed by shaping and welding a flat configuration as described above, but is preferably formed by directly cutting it from tubular stock, such as by laser cutting, for example.

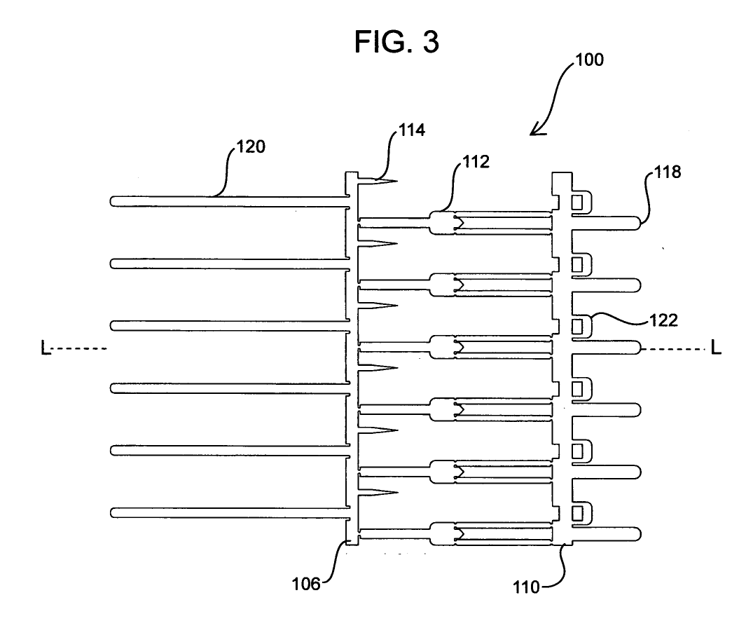

FIG. 3 shows a flat pattern of another example of an anastomosis device 100 according to the present invention. Like device 1, device 100 is generally formed integrally, as an annular structure, for example, by laser cutting from tubular stock, although it would be possible to cut or stamp a planar structure from a sheet of material and then weld or otherwise fix the device in its annular form, or otherwise cut the pattern from tubular stock. Device 100 may be made from the same materials as described with regard to device 1.

In this arrangement, only two rings 106, 110 are provided to form the basic cylindrical structure of device 100. Buckling struts 112 join rings 106 and 110 and are generally equally spaced around the circumferences of the rings 106 and 110 to form a buckling portion of the device 100. Buckling struts 112 are bent outwardly from an outer surface of an imaginary cylinder defined by rings 106 and 110, to make the buckling portion more susceptible to collapse upon exertion of compressive forces along the longitudinal axis of device 100 and to direct the buckling motion of struts 112 in an outward direction so as to effectively increase the outside diameter of the buckling portion upon buckling. Graft tines 114 extend from ring 106, and are bent to positions substantially perpendicular to the longitudinal axis of device 100 during forming, to position them for anchoring the end of a graft, as discussed further below.

External tines 118 extend from ring 110 and are bent substantially perpendicularly to the longitudinal axis L of device 100 during forming. Locking tines 120 extend from ring 106 at substantially evenly spaced locations about the circumference of ring 106. Locking tines 120 have a sufficient length to span the remaining length of device 100 when they are folded over by one hundred and eighty degrees during forming. Locking receptacles 122 are formed adjacent external tines 118 and extend from ring 110 in alignment with locking tines 120, and are bent substantially perpendicularly to the longitudinal axis L of device 100 to allow locking tines 120 to pass therethrough during formation of the device. The locking tines 120 are bent over to the external side of the general cylindrical shape of device 100, and threaded through the locking receptacles 122. By passing locking tines 120 through receptacles 122, locking tines 120 effectively link rings 106 and 110 to provide an important locking feature upon deployment of the device, as will be discussed below. External tines 118, along with the locking tines, when they are bent over during the locking procedure, form contact surfaces by which device 100 applies pressure to the external surface of a vessel (e.g., external wall of the aorta) to which a graft held by device 100 is being joined. Although not shown, an alignment tab 124, such as shown in the device 1 may be included on device 100, either adjacent to, or in place of one of external tines 118, to control proper alignment of device 100 when loaded on a deployment instrument.

Deployment Instrument

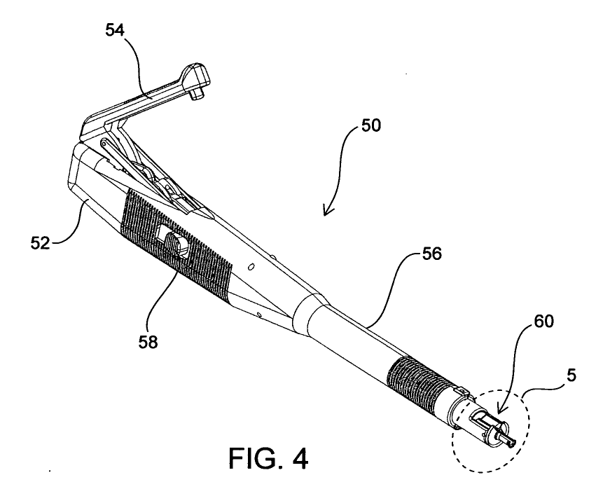

FIG. 4 is a perspective view of a deployment instrument 50, which is configured to receive and deliver an anastomosis device in performance of an end-to-side anastomosis. Generally speaking, instrument 50 includes a main body or handle portion 52, which is configured to be hand held by the operator. A distal tip portion 60 of instrument 50 is configured for receiving, holding and deploying an anastomosis device 1,100 according to the present invention. A driving 1 ever or trigger 54 is actuated by squeezing to move it towards handle 52 to perform a deployment of an anastomosis device. A long, slender extension portion 56 separates the distal tip portion 60 from the handle 52 by a sufficient distance to adapt the device to be employed in vary small spaces and even endoscopically in some situations. The handle 52, trigger 54 and extension 56 may all be formed of a structurally rigid polymer, such as ABS plastic or other materials which are sufficiently rigid and biocompatible.

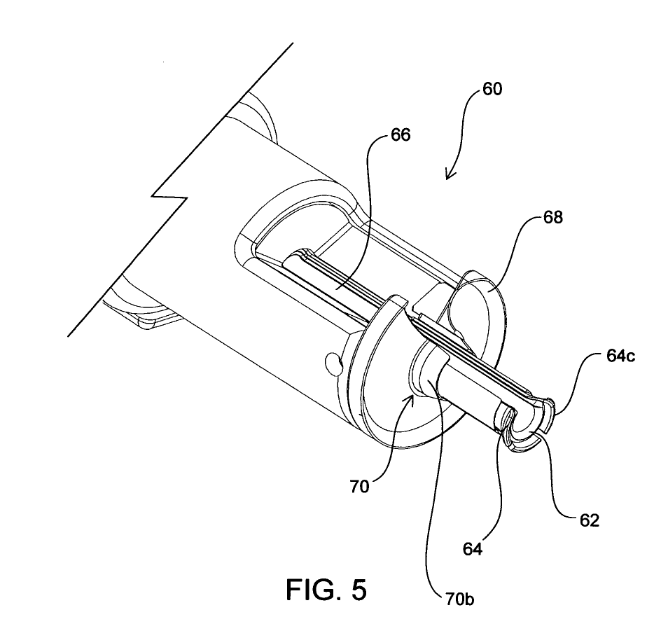

FIG. 5 is an enlarged view of the distal tip portion 60 of instrument 50, which is enlarged from the section delineated by phantom circle 5 in FIG. 4. Distal tip portion 60 includes an assembly of substantially cylindrically shaped tubes, which are concentrically arranged for receiving, holding and deploying an anastomosis device. Of course, those of ordinary skill in the art would recognize that the assembly of tubes could be formed with other conforming cross-sectional shapes, for example, elliptical, oval or other cross-sectional shape tubes could be substituted. The outside diameter of the arrangement is slightly less than the inside diameter of a device 1,100 for which it is designed to receive and deploy. For example, a clearance of about 0.002″ may be provided between the inside diameter of the clip 1, 100 and the outside diameter of the arrangement. Such a design allows the device 1,100 to be freely slid over the tube portions when in the loading configuration, while at the same time not allowing so much clearance as to allow the device to become misaligned. Because of this fairly close tolerance requirement, instruments 50 having varying distal portion outside diameters are manufactured to match the inside diameters of the various device sizes that may be needed. As discussed above, the device sizes may vary in the range of about 3.0 mm to about 7.0 mm inside diameter, which necessitates the provision of a series of delivery instruments 50 to accommodate the size variations.

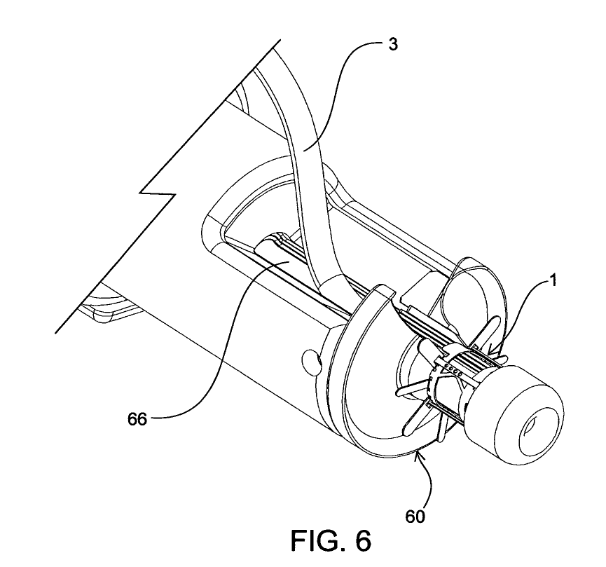

Each of the concentric tubes is provided with a longitudinal slot so as to define a channel 66 in the top of the arrangement that allows a graft (attached to a device 1, 100) to extend externally of instrument 50, and to render the cross-sectional views of the tubes to appear somewhat “C-shaped”. Advantageously, this feature allows a graft to be side fed into instrument 50 and also does not require that both ends of the graft be free in order to perform an anastomosis according to the invention. For example, FIG. 6 shows a graft 3 fixed to a device 1 and the device having been captured on distal portion 60 of instrument 50 for performing a proximal anastomosis of graft 3 with an aortic wall. In this case, an internal mammary artery was used as the graft and so the opposite end of the graft (not shown) is still connected to the vasculature of the patient. Further, this feature would also allow the distal anastomosis of a graft initially having two free ends (such as a saphenous vein graft as one, non-limiting example) prior to the proximal anastomosis of the graft.

Currently known procedures using mechanical anastomotic coupling typically require the proximal anastomosis to be performed before the distal anastomosis is performed. This is disadvantageous for at least two reasons. One reason is that surgeons are currently trained to perform the distal anastomosis prior to performing the proximal anastomosis. A second reason is that, depending upon the location of the coronary artery which is being bypassed, it is very frequently necessary to move the heart out of its natural position, such as by elevating it out of the chest cavity to provide access to the site where the anastomosis is to be performed. If the proximal anastomosis must be performed first, this makes it very difficult, if not impossible to accurately measure the length of graft that will be needed to properly perform the distal anastomosis. This is so, because in the displaced position, the heart is not fully perfused, and therefore any measurements made at this time are almost certain to be inaccurate, as the actual distance between proximal and distal anastomosis sites will change when the heart is returned to its natural position and becomes fully perfused, thereby enlarging somewhat. The current invention allows the distal anastomosis to be performed first, after which the heart can be properly positioned and an accurate assessment of the graft length needed can be made before performing the proximal anastomosis.

Therefore, it is often advantageous to perform the distal anastomosis prior to the proximal anastomosis in a cardiac bypass procedure as it is much easier to gauge the correct length to which the graft needs to be cut when the distal anastomosis is performed first since the heart will be normally loaded with blood and the surgeon can get a better approximation of where the locus of the proximal anastomosis will reside after completion of the procedure, which allows a more direct measurement of the length of the graft needed. As noted, the heart very often needs to be displaced to perform the distal anastomosis. By performing the distal anastomosis first, the heart can then be repositioned to its natural location and orientation, thereby making it much easier for the surgeon to visualize and directly measure or approximate the length of graft needed to reach the proximal anastomosis site. Since most surgeons traditionally perform the distal anastomosis first, even when using suturing methods, they will be more inclined to accept a procedure where distal anastomosis can be performed first.

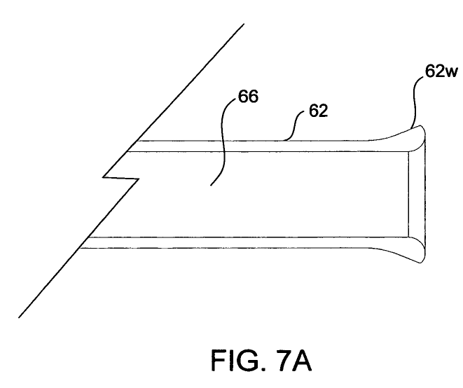

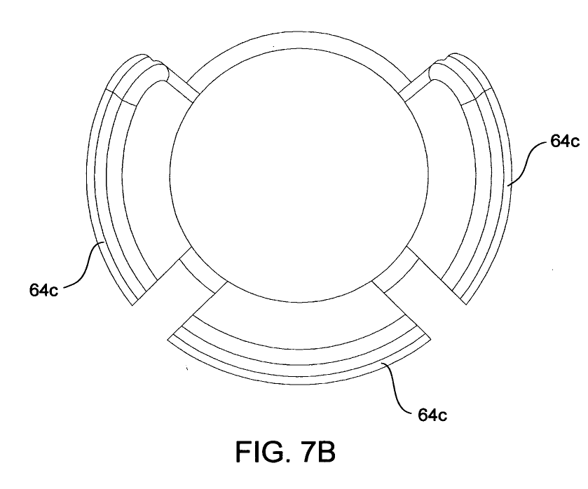

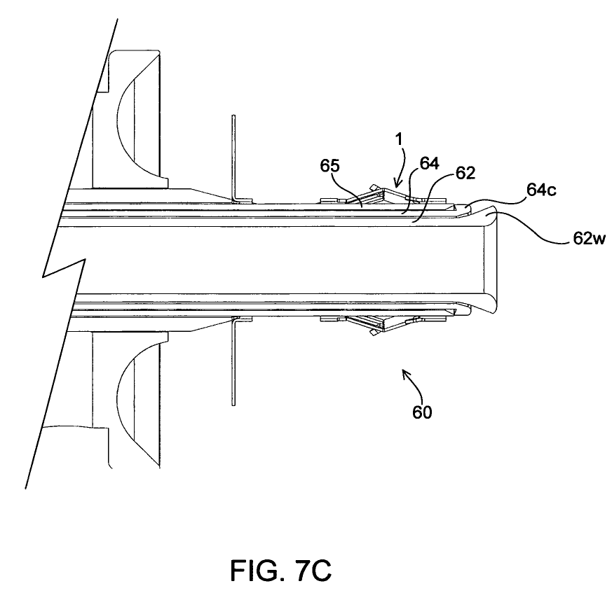

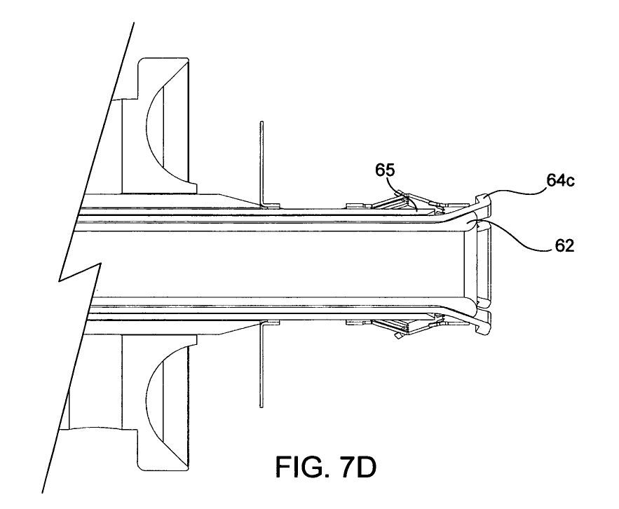

The concentric tube arrangement includes a wedge tube 62 concentrically surrounded by a catch cam tube 64, with these tubes arranged for relative sliding movement with respect to one another along their longitudinal axes. A release tube 65 is concentrically arranged over catch cam tube 64, and is relatively fixed to wedge tube 62 so that is slides relative to catch cam tube 64 when wedge tube 62 is slid relative to catch cam tube 64. The wedge tube 62, catch cam tube 64, and release tube 65 operate in conjunction with other features of the instrument 50 to perform the functions of capturing an anastomosis device 1,100; buckling the device; locking of the device; and finally releasing the device from the distal portion 60 of instrument 50. An anastomosis device is securely mounted or loaded onto the distal portion 60 of the instrument 50 by way of the capture function. The wedge tube 62 includes a flared or wedged end portion 62w that has a generally increasing outside diameter as shown in FIG. 7A. Catch cam tube 64 has an inside diameter that is freely slidable over the outside diameter of the non-flared portion of wedge tube 62, and is split or slotted at its distal end to form a plurality of expandable fingers or catches 64c (e.g., three are shown in the end view of FIG. 7B, although 1, 2 or 4 or more could be formed).

FIGS. 7C-7D show the interaction between wedge tube 62, catch cam tube 64 and release tube 65 during a capture procedure for fixing a device 1,100 on the distal portion 60 of the deployment instrument 50. It is contemplated that the capture, compression and release functions described herein could be accomplished by a device as described, but which lacks a release tube 65 as described. However, it is has been found that the instrument 50 operates more smoothly and reliably with the release tube 65 for reasons described below. Initially, a device 1,100 is slid over the concentric tubes 62,64 when the instrument is in the neutral or loading position as shown in FIG. 7C, such that the wedged end 62w of wedge tube 62 extends beyond the catch cam tube 64 and does not make contact with catch cams 64c. The release tube 65 surrounds the catch cam tube 64 in this configuration and ensures that the catch cam members 64c are positioned in their fully retracted configuration. The deployment device 50 is placed into the neutral position by advancing a pin or button actuator 58 (see FIG. 5) located on the side of handle 52. Advancement of the button or pin 58 pushes a central shaft that is connected to wedge tube 62, which advances wedge tube 62 so that the wedge portion 62w extends beyond catch cams 64c and therefore does not make contact with them, allowing the catch cams 64 to retract to a resting configuration in which the outside diameter of the end of the catch cam tube 64 (formed by catch cams 64c) is smaller than the inside diameter of a device 1,100 to be loaded thereon. Thus, the catch cam tube is in a relaxed or retracted configuration and even the catch cams have a smaller outside diameter than the inside diameter of the device 1,100 to be captured. For this reason, device 1,100 is freely slidable over the tubes 62,64,65.

Device 1,100 may include an alignment tab or tine 24 extending from ring 1,100 which is bent over, radially inward of the device into an orientation substantially perpendicular to the longitudinal axis of the device L during forming. Device 1,100 is aligned with instrument 50 by sliding alignment tab 24 in channel 66. This alignment ensures that each of the locking tines 20,120 will be properly aligned so as to be contacted by device lock 68 during the locking operation described below. The device 1, 100 is slid onto the distal portion until it makes contact with stop member 70. Stop member 70 is fixed with regard to handle 52 of device 50. Stop member 70 may include a beveled portion 70b, which provides a ramping surface against which device 1,100 comes to rest. In this way, stop member not only correctly positions device 1,100 in a longitudinal position along the distal portion 60, but also performs a centering function to keep device 1,100 properly centered on the distal portion 60 of deployment device 50.

Once device 1,100 is properly positioned and abutted against stop member 70, pin or button 58 is released, and wedge tube 62 is spring loaded so as to be drawn back with respect to catch cam tube 64, such that wedge portion 62w slides against and contacts catch cams 64c, radially expanding them to assume a larger outside diameter, as shown in FIG. 7D. At the same time, release tube 65, which is linked to wedge tube 62, retracts so that it no longer prevents the expansion of the catch cams 64c. Catch cams 64c, when in the expanded or deformed position, form a larger outside diameter than the inside diameter of device 1,100 and therefore capture device 1,100 on the distal tip portion 60 since device 1,100 is prevented from sliding off distal tip portion by the hooked configurations of catch cam 64c.

Device 1 is securely held by the abutment of ring 10 against stop member 70, and by contact of ring 6 by catch cams 64c.

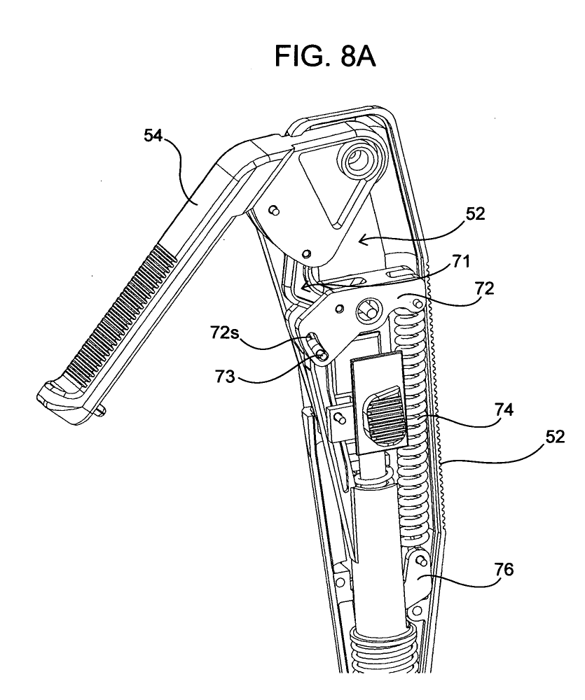

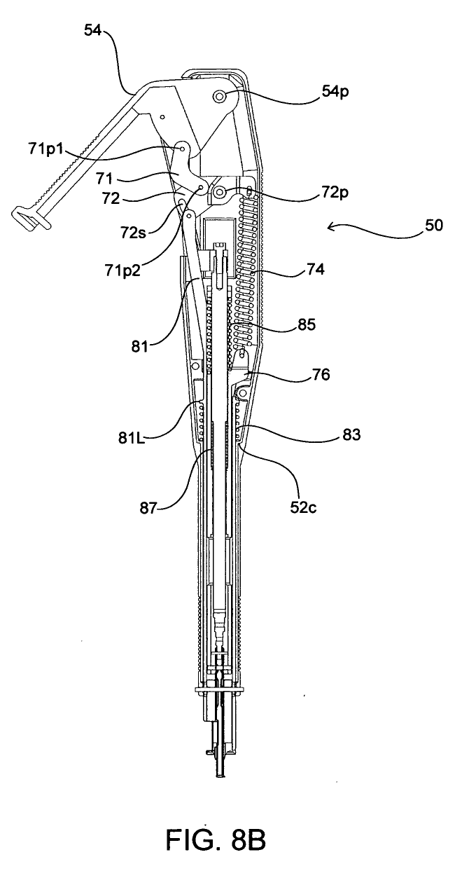

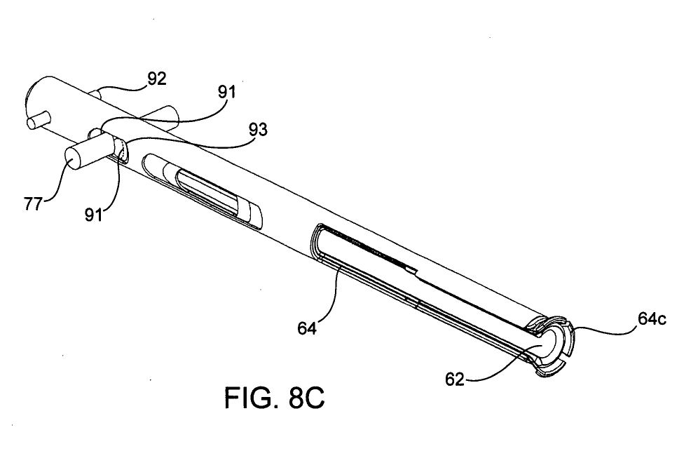

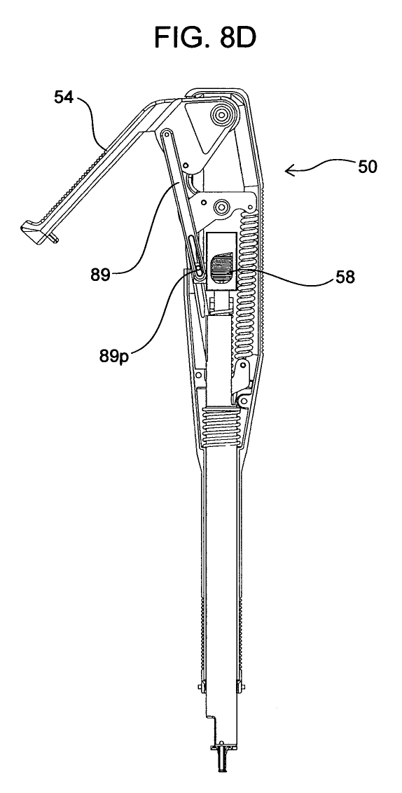

FIGS. 8A-8D are views of the internal components of deployment device 50 which link trigger 54 with various components at the distal end portion 60 of device 50 for performing the capture, buckling, locking and release functions during performance of an anastomosis.

Referring to the proximal end portion view of FIG. 8A, a four bar linkage arrangement is provided in the form of trigger 54, trigger link 71, rocker 72 and the handle 52 of device 50. As the trigger 54 is pulled or pressed toward handle 52, it drives trigger link 71, which in turn drives rocker 72 in rotation toward the distal end of device 50, causing a retraction of catch cam tube 64 through extension spring 74 which is connected to compression slider 76 that connects through the catch cam with pin 77 (see FIGS. 8B and 8C), in a direction toward the proximal end of device 50. At the same time, stop member 70 remains fixed relative to device 1,100, resulting in a compression force being applied to device 1,100 as catch cam tube 62 retracts. Wedge tube 62 is spring loaded with respect to catch cam tube 64 by way of a compression spring extending between pin 92 (which interlinks wedge tube 62 and release tube 65) and compression slider 76 (which is connected to catch cam tube 64 in the manner described above), so that, in their resting positions, wedge tube 62 is biased in extension relative to catch cam tube 64 which allows the catch cams 64c to relax or retract. A slot 91 is provided in the wedge tube 62, as shown (in phantom) in the isolated assembly of FIG. 8C. A longer slot 93 is formed in the release tube 65. Pin 77 goes through slot 91 and is retained by a through hole in catch cam tube 64 which forms a press or friction fit with pin 77 as it is positioned through slot 91 and the hole. In the view shown in FIG. 8C, the wedge tube 62 has been retracted so as to expand catch cams 64c and pin 77 is positioned against the proximal end of slot 91. This occurs during the retraction by compression slider 76, which overcomes the compression spring, thereby deforming it under a compressive load, and allowing wedge tube 62 to move proximally with respect to catch cam tube 64 until pin 77 abuts the proximal end of slot 91. By this arrangement, further retraction for compression of a device 1,100 results in the catch cam tube 64 and wedge tube 62 sliding proximally in unison, to ensure that the catch cams 64c remain in the expanded configuration, thereby ensuring that the capture of device 1, 100 is maintained during compression.

Pin 77 can slide in the slot 91 on reverse motion to allow the catch cam catches 64c to retract as the wedge 62w extends distally of them, then the pin 77 contacts the distal (opposite) end of the slot 91 so that the catch cam tube 64 and wedge tube 62 again move together in any further distal sliding. That is, when the tension on spring 75 is relieved so that it no longer draws against compression slider 76, as catch cam tube returns to the reset position, the compression spring between pin 92 and compression slider 76 extends to release its compression, thereby sliding wedge tube 62 distally with respect to catch cam tube 64 until pin 77 contacts the distal end of slot 91. This biasing by the compression spring maintains the catch cams 64c in their retracted configuration in the reset position of device 50. During the compression motion, as the catch cam tube 64 and the wedge tube 62 are proximally slid in unison, the catch cams 64c and stop 70, a sa result, compress device 1,100 so that initially, the buckling section of the device buckles. Thus, in the case of device 1, the buckling section between rings 6 and 8 collapses or buckles first with struts 12 moving radially outwardly during buckling, as described above, to form a mushroom-shaped configuration.

As the trigger 54 continues further in its travel toward the body 52, the struts 16 of the strut section begin to collapse as the catch cam tube 64 and wedge tube 62 further advance toward stop 70. The collapse of the strut section is accomplished to draw a graft and vessel together during an anastomosis procedure with a sufficient force to form a successful seal between the two, while not compressing the anastomosis with too great a force to potentially cause damage to the living tissue. As such, the collapse of the strut 16 draws the rings 8 and 10 closer together, which effectively also draws the buckled struts 12 closer to ring 10, thereby compressing the tissues which are held there between during an anastomosis procedure.

Extension spring 74 interconnects rocker 72 with compression slider 76, which retracts the catch cam tube as described above. Extension spring 74 acts as a force limiter during the compression/buckling stage. Extension spring 74 has a preset load at which it begins to expand. F or example, extension spring may be designed so that the coils do not begin to expand or separate until a load of about 20 pounds has been reached. The effect achieved by this is that the catch cam tube will continue to be retracted, and therefore continue to compress/buckle device 1,100 until such time as a 20 pound load is exerted upon the extension spring 74, or until rocker 72 goes over center and reverses direction (via the 4 bar linkage. When an imaginary straight line connecting the two pivot points 71p1 and 72p2 becomes parallel with an imaginary straight line interconnecting trigger pivot 54p and rocker pivot 72p, the four bar linkage is considered to be at “center”. Further driving by the trigger 54 causes the linkage to go over or beyond center, which drives rocker 72 into a reverse rotation. The preset load on the extension spring may be reached or achieved when the buckled struts 12 (which carry an everted graft end) and external tines 18 compress the tissues there between sufficiently to form a leak tight seal.

Once the predetermined force or load is reached, extension spring 74 begins to extend, so that no further driving/retraction of the catch cam tube 64 can occur and device 1,100 is therefore compressed no further. For example, accounting for about 8-9 pounds required to buckle a device, 1,100, and the force need to counteract a reset spring 85, which abuts against the handle 52 and the compression slider 76 to exert a return or resetting biasing force to reset the catch cam when no force is being applied to it by spring 74 of the deployment device, an extension spring 74 having a preset load of about 20 pounds translates to a compression force of about 3-4 pounds which is actually applied to the tissues compressed by device 1,100 when spring 74 begins to extend. Of course the present invention is not limited to a final compression force of about three to about four pounds, as slightly less force may be applied (e.g., about one to three pounds) or slightly greater force, so long as it is not so great as to cause tissue damage.

With the force-limiting feature, device 1, 100 is not collapsed to a predefined length. Rather, it is collapsed until a predefined buckling force is achieved. Because of this, device 1,100 can reliably seal an anastomosis of a graft to vessels of varying wall thickness, wherein the compressive force for connecting a graft to a thin walled target vessel (e.g., aorta) is substantially the same as the compressive force established when connecting a graft to a thick walled vessel (e.g., an aorta having a relatively thicker wall than the previous one). That is, instead of forcing the device 1,100 into a particular thickness, it is adjustable to various wall thicknesses, and is controlled to be collapsed only to a thickness that will achieve a predetermined amount of compressive force on the site of the anastomosis. Practically speaking, this means that the thickness of the gap in which device 1,100 compresses the graft and vessel will vary with the thickness of the vessel wall and graft wall, but will achieve substantially the same compressive force regardless of the thickness of the tissues being joined.

As the trigger 54 continues its motion toward the handle/body 52, after the buckling of device 1,100 has been accomplished, pin 73 reaches the end of slot 72s in rocker 72. Continued advancement of rocker 72 then drives lock driver 81, which is an integral portion of (or may be connected to) device lock tube 81 (upon which the device lock 68 is fixed) at its distal end As the device lock tube 81 is driven in a direction toward the distal end of deployment device 50, this motion drives device lock 68 toward device 1,100, while catch cam tube 64 and wedge tube 62 remain fixed with respect to device 1,100. Additionally, a lock spring 83 which abuts a ledge or shoulder 81L formed on device lock tube 81 at one end, and another ledge, abutment or shoulder 52L formed in handle 52, is compressed by the advancement of device lock tube 81 relative to handle 52. Stop member 70 is fixed with regard to handle 52, and therefore maintains its fixed position as device lock tube 81 and device lock 68 advance. The device lock 68 includes curved guide surfaces 68g which guide the ends of locking tines to be bent radially outward, with further advancement of device lock 68 bending the locking tines 20,120 over locking receptacles 22,122 and against external tines 18 or the wall of the graft (in the case of a design such as device 100). By bending the locking tines 20,120 over against locking receptacles 22,122, the locking tines secure the positions of rings 6 and 10 from being spread apart. This permanently sets the positions of the rings and the force applied thereby, preventing the device 1, 100 from expanding or unbuckling.

As the trigger 54 completes its travel toward handle 52, the reverse rotation of rocker 72 releases the force between rocker 72 and device lock tube 81, which allows the biasing force contained in lock spring 83 to reset the tool. The locking driver (device lock) 68 is retracted back to its neutral starting position, thereby breaking contact with the locking tines 20,120. At the same time, the reverse rotation of the rocker 72 takes the load off spring 74 so that the biasing force of spring 85 drives the compression slider 76 and catch cam tube distally to their neutral positions. The wedge tube 62 is driven distally along with the catch cam tube 64. The motion of the trigger 52 going forward (i.e., toward the body of the tool) also drives wedge link 89, so that an end of the slot 89s in wedge link 89 abuts pin 89p connected to button 58, and then drives button 58 distally to further drive the wedge tube 62 in the distal direction so that the wedge portion 62w breaks contact with catch cams 64c, which, as a result, return to their relaxed or retracted positions, to define an outside diameter that is smaller that the inside diameter of the device 1,100. This is the release position of the deployment tool, and allows the distal end portion 60 to be slid out from inside device 1,100, leaving device 1,100 undisturbed at the site of the anastomosis.

Although the catch cams 64c retract to a conformation that may be slid out from inside the device 1,100, it was discovered that there was still some potential for one or more of the catch cams 64c to catch on a ring or strut of the device 1,100 as the deployment tool 50 was being withdrawn. For example, if the device 1,100 was allowed to drop down on the distal end portion 60, this would leave a large gap between the deployment device end portion 60 and the bottom of the device 1,100, while the top portion of device 1,100 would contact the catch cam tube 64 and then be trapped by the catch cam 64c during an attempt to remove the deployment tool. To ensure that the deployment tool 50, and particularly a catch cam 64c does not catch on the device 1,100 during removal of the tool 50, a release tube 65 is provided, as shown in FIGS. 7C and 7D.

Release tube 65 concentrically surrounds catch cam tube 64 for sliding movement relative thereto, and also has a slot to match those of the catch cam tube 64 and wedge tube 62. Release tube 65 is linked to wedge tube 62, such as by a pinned interlink 92, so that it moves together with wedge tube 62 at all times. Thus, during the loading/capture of a device 1, 100, release tube 65 is retracted away from the catch cams 64c as wedge 62w is retracted into the catch cams 64c to expand them (as shown in FIG. 7D). This removes the release tube from the vicinity of the catch cams 64c allowing the catch cams 64c to more effectively capture the device 1,100.

During the release procedure, as the wedge tube 62 is pushed out from the catch cam tube 64, release tube 65 slides with wedge tube 62, so as to approximate the catch cams 64c of catch cam tube 64, as shown in FIG. 7C. Although FIG. 7C shows loading a device 1,100 onto the tool 50, the release tube 65, catch cams 64c and wedge 62 are positioned the same as when a release of the device 1,100 is being performed. Release tube 65 also may function to slightly compress the fingers of the catch cams 64c by a force opposite to that that is applied by the wedge tube when the fingers are expanded. Release tube 65 is dimensioned such that the external surface of the tube extends slightly higher than the extent of catch cams 64c. Therefore, when deployment tool 50 is removed from device 1, 100, even if the device 1,100 does drop down, it slides along the surface of release tube 65 and clears the catch cams 64c providing for a smooth removal of the deployment device.

Performing the Anastomosis

The present invention is applicable for performing a variety of anastomosis procedures, including coronary artery bypass grafting. One or more anastomoses are performed on a target vessel within a patient, by connecting one or both ends of a graft to the target vessel. The following description pertains to a specific, non-limiting application of the present invention in performing an end-to-side anastomosis of a proximal end of a graft to the wall of the aorta.

The description begins with the surgical site having already been prepared for performance of the anastomosis. The anastomosis can be performed with the heart stopped and the patient on cardiopulmonary bypass or during a beating heart bypass procedure. Examples of grafts appropriate for use in performing an anastomosis include an internal mammary artery having only one free end (the end on which the anastomosis is to be performed), a saphenous vein graft or radial artery graft having two free ends (in which case it is possible to perform the distal anastomosis first, if desired, as noted above) or some other suitable graft or conduit.

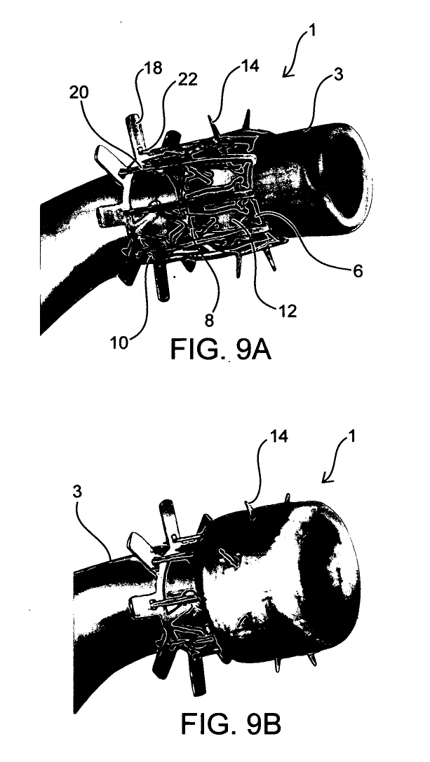

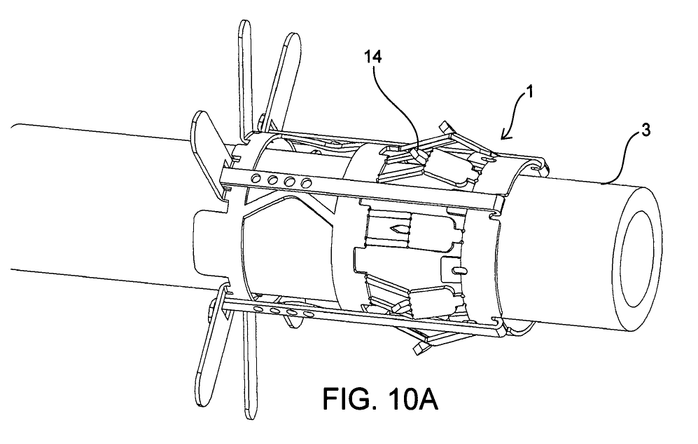

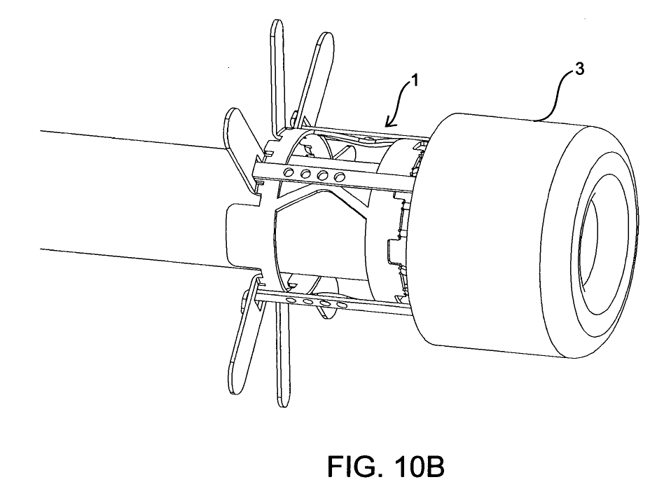

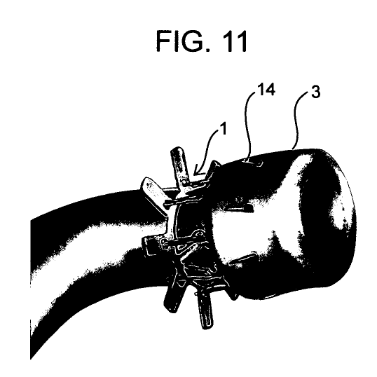

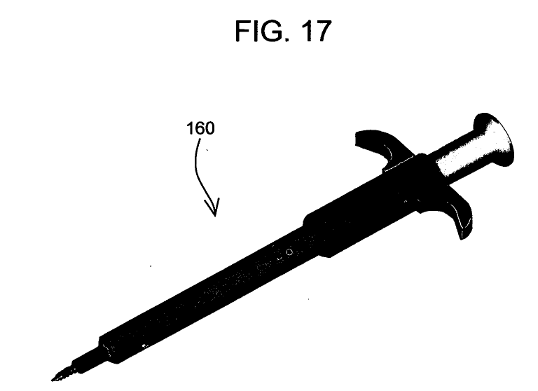

After selection and preparation of the graft to be used, the proximal end of the graft 3 is loaded and everted onto the device 1, by passing the proximal end 3 through the interior of the device 1,100 and then everted over the proximal end of the device 1,100, as shown in FIGS. 9A-9B and 10A-10B. In the case of FIGS. 9A-9B, where elongated graft tines 14 are employed, such as with the device 1 of FIG. 1A, or with device 100 of FIG. 3, the tines 14 pierce and extend through the wall of the graft 3 as shown in FIG. 9B. In this situation, the tines are preferably further bent over, after the eversion, as shown in FIG. 11 to facilitate insertion of the graft 3 and device 1,100 through the opening in the target vessel for performance of the anastomosis. In the case of a device 1,100 which uses the shortened tines (such as the device 1 shown in FIG. 1B, for example) or which uses no tines at all, tines do not extend through the wall of the vessel 3 upon performance of the eversion, as shown in FIG. 10B. The shortened tines 14 pierce into the wall, but do not extend through and out of the wall. When no tine are used, the appearance is the same as shown in FIG. 10B. FIG. 6 shows the graft 3 having been loaded on a device 1,100 and onto a deployment tool 50. As described above, by advancing button 58 distally, the wedge 62w of wedge 62 extends beyond catch cams 64c, thereby allowing device 1,100 (along with graft 3) to be mounted on the tool 50. The concentric tubes 62,64,65 of the distal end of tool 50 are inserted between the graft 3 and device 1,100. The portion of the graft which extends in the direction of the trigger 54 can be positioned within channel 66, as shown in FIG. 6. Once the graft 3 has been loaded and everted on a device 1,100 and device 1,100 has been captured by deployment tool 50, an aortotomy punch 160 as shown in FIG. 17 (available from Guidant, Santa Clara, Calif.) or other cutting or punching instrument is used to punch a hole in the wall of the aorta at the site that the anastomosis is to be performed.

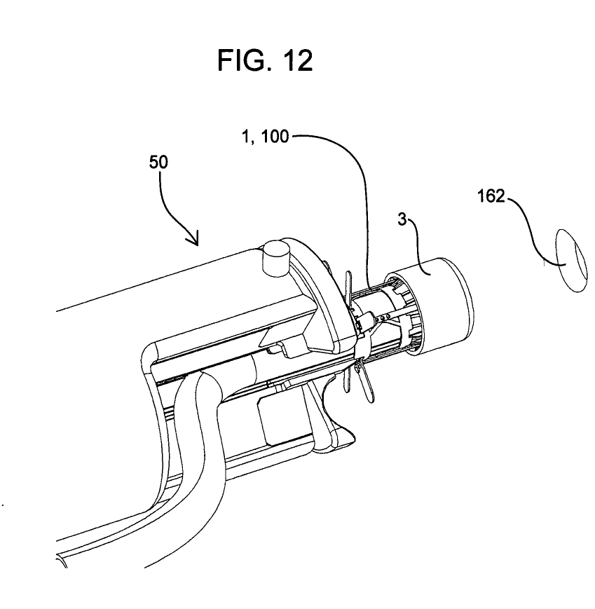

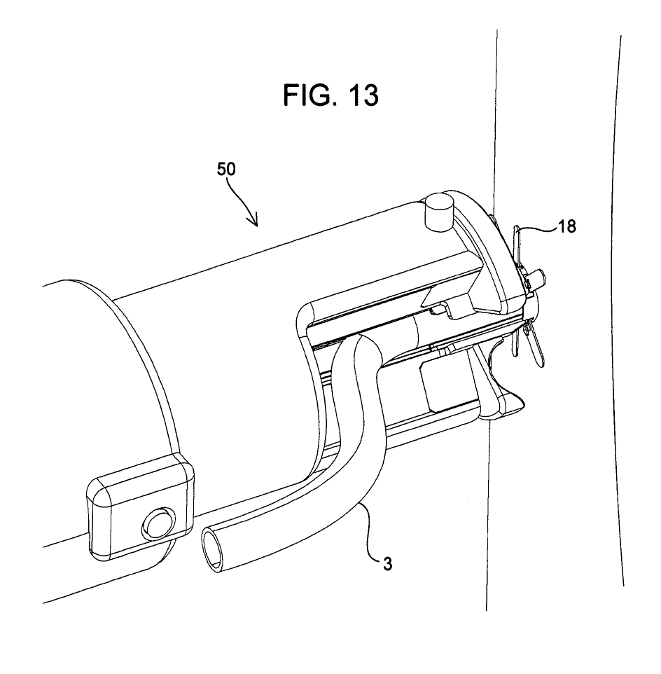

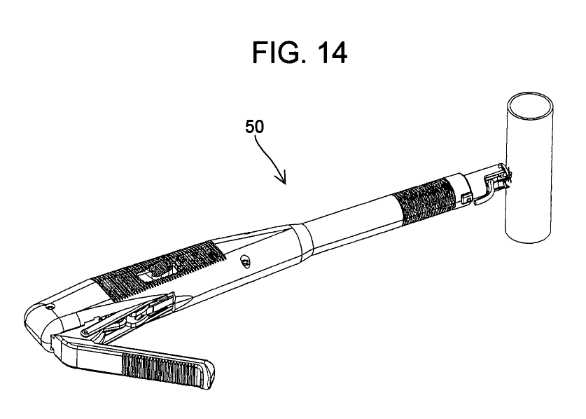

Aortotomy punch 160 provides an initial blade stab with a retracting rotary punch that creates a circular aortotomy 162 having a specific diameter that is matched to the outside diameter of the graft 3 everted over the device 1,100, see FIG. 12. For a beating heart procedure, the aortotomy is temporarily sealed, such as by application of finger pressure by the surgeon, to prevent blood loss while the graft assembly is approximated to the aortotomy 162. The finger pressure is then released and the graft/device are inserted into the aortotomy, as shown in FIGS. 13 and 14, preferably using a rolling or rotating motion which allows a rapid insertion to stop the majority of blood flow from the aortotomy 162. The graft/device are inserted until the external tines 18 abut the external wall of the aorta, at which time the deployment of the device begins.

With a single continuous squeeze or depression of the trigger 54 toward the handle 52 of the deployment tool 50, the device 1,100 is compressed, compression fitted and locked to join the graft 3 to the aortic wall, and the deployment tool 50 then releases its capture of the device 1,100 so that the surgeon can remove the deployment tool from inside the device 1,100 with the graft 3 at the same time being slid out of the channel 66, thereby completing the anastomosis.

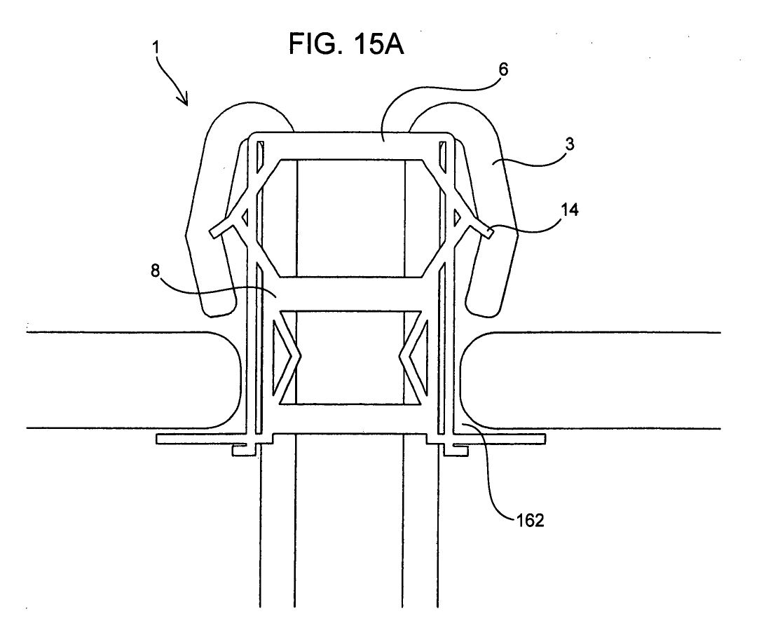

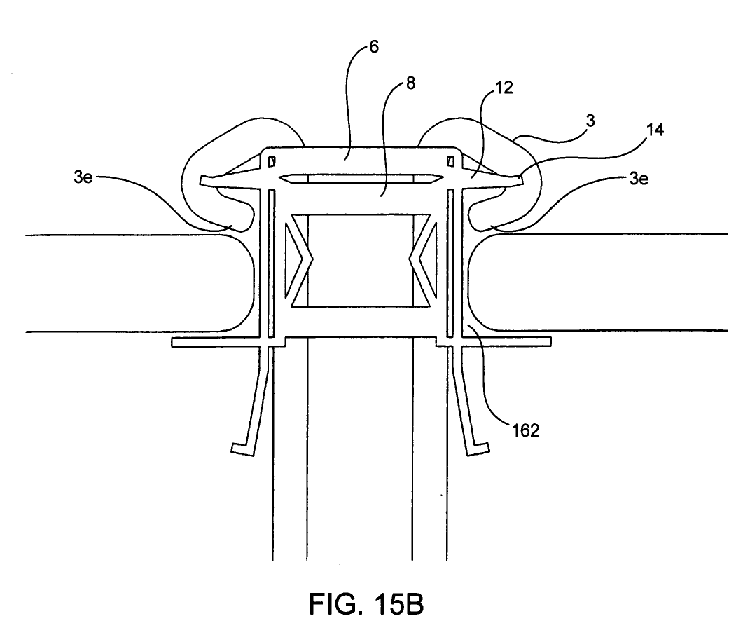

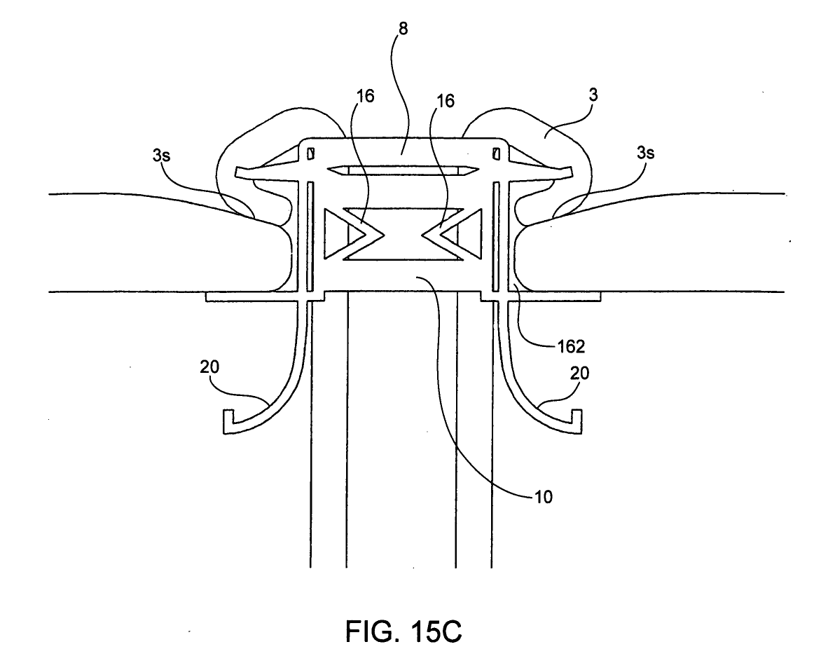

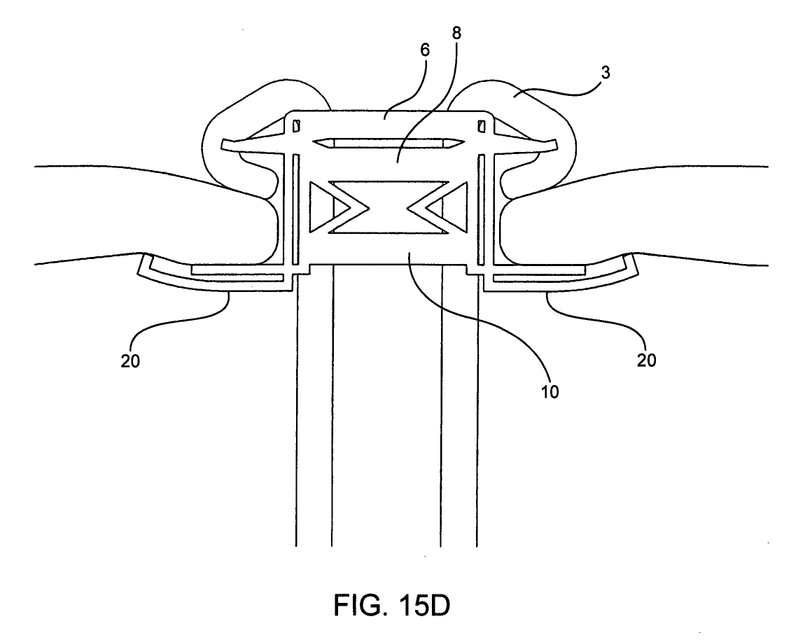

FIGS. 15A-15D schematically show the various stages of buckling, compressing and locking that are performed in rapid succession during a single pull of the trigger 54. For purposes of clarity, the deployment device has not been shown in FIGS. 15A-15D. In FIG. 15A, the graft 3 and device 1 are shown just after insertion into the aortotomy 162 and prior to squeezing the trigger 54. Initially upon pulling the trigger 54, the retraction of catch cam tube 64 first causes the buckling section between rings 6 and 8 to collapse or buckle, as shown in FIG. 15B. Due to the partially bent configuration of the struts 12, a controlled direction of buckling is assured which causes a mushroom-shaped configuration to result as shown. The buckled configuration of the buckled struts 12 forms an internal retraining structure, which is drawn to provide a compression force of the graft tissue against the internal aortic wall. The shape and direction of buckling of struts 12 are advantageous in that they further evert the proximal end of the graft at 3e so that the intima of the graft 3 approximates the intima of the aorta in preparation for forming an intima to intima anastomosis. The further eversion 3e of the graft also assures that there will be no metal contacting either the intima of the aorta or the intima of the graft at the site of the anastomosis, thereby assuring a more reliable seal and more reliable healing.

As the trigger 54 continues further in its travel toward the body 52, the struts 16 of the strut section begin to collapse, as shown in FIG. 15C, as the catch cam tube 64 and wedge tube 62 further advance toward stop 70. The collapse of the strut section draws the graft 3 and aorta together with a sufficient force to form a successful seal 3s between the two, while not compressing the anastomosis with too great a force to potentially cause damage to the living tissue. As such, the collapse of the struts 16 draws the rings 8 and 10 closer together, which effectively also draws the buckled struts 12 closer to ring 10, thereby compressing the everted face 3e of the graft and the wall of the aorta. As noted earlier, the extension spring 74 of the deployment device acts as a force limiter, so that the struts 16 are collapsed only so far as to establish a predetermined compression force between the graft 3 and the aorta. In this way, the struts 16 define a compression zone, the length of which is adjustable to provide a predetermined compression force to varying thicknesses of target vessel.

As the trigger 54 continues its motion toward the handle/body 52, and the lock driver 81 is driven in a direction toward the distal end of deployment device 50, the device lock 68 bends over the locking tines 20, as shown in FIG. 15D, thereby firmly locking the relative positions of the rings and 6, 8 and 10, to set the compression force maintaining the anastomosis. The locking tines may be provided with sharp points, barbs, or other configuration at their distal ends to facilitate piercing or other mechanical engagement of the outer wall of the aorta. As the trigger 54 completes its travel toward handle 52, the device lock 68 is retracted back to its neutral starting position, thereby breaking contact with the locking tines 20, and the wedge tube 62 is driven distally so that the wedge portion 62w breaks contact with catch cams 64c, which return to the relaxed position, to define an outside diameter that is smaller than the inside diameter of the device 1. This is the release position of the deployment tool 50, and it allows the distal end portion 60 to be slid out from inside device 1, and the graft 3 is slid out of the groove 66, leaving device 1 and graft 3 undisturbed at the site of the anastomosis.

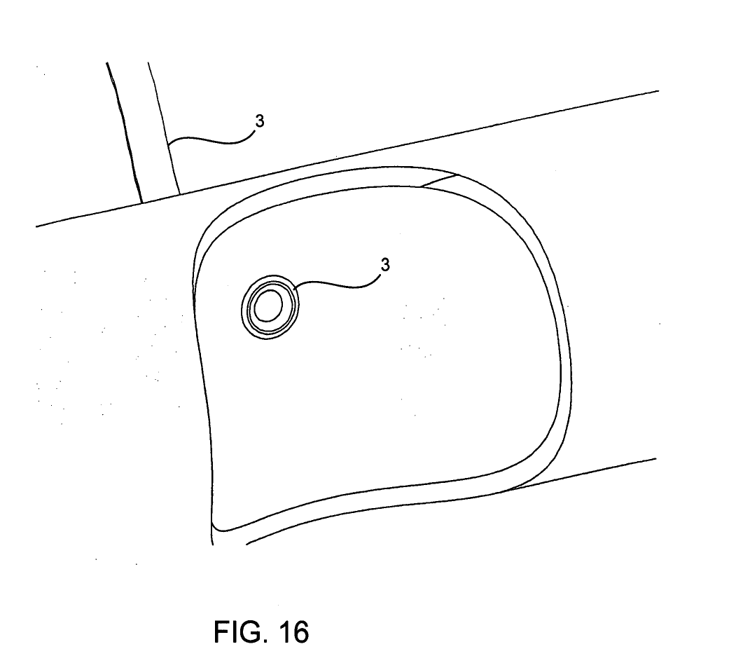

Device 100 is deployed in the same manner as described above with regard to device 1. However, with only one set of struts 112, the struts expand outwardly by a greater distance and expand beyond the extent of the everted end of the graft 3. Additionally, since the graft tines are located on the ring 106, the graft 3 is not everted to as great an extent as what occurs when buckling the device 1. The result is still an intima to intima anastomosis, but the intima to intima contact is periodically interrupted by the radially extending collapsed struts 112 which extend therebetween. For this reason the device 1 is preferred. FIG. 16 is a top view of a completed anastomosis viewed on the inside wall of a target vessel, where device 1 of FIG. 1B was used to perform the anastomosis. Only the everted graft may be seen and only the everted graft tissue contacts the wall of the target vessel where the seal between the two is formed. With no exposed metal or any portion of device 1 extending from the jointure of the graft and the target vessel, the resultant anastomosis greatly improves the opportunity for healing and growth between the two joined tissue components, and reduces the risk of leakage, clotting, or other deposits which might tend to form on exposed metal.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A device for use in making an anastomosis between tubular fluid conduits in the body of a patient, said device comprising a unitary structure having a main body disposed annularly about a longitudinal axis and having first and second end portions; a plurality of members extending radially outwardly from said first end portion; and said second end portion having a plurality of spaced struts adapted to buckle in a radially outward direction upon axial compression of said device.

2. The device of claim 1, wherein said first end portion further comprises a second set of spaced struts, said second set of spaced struts being collapsible over a variable range of distance to accommodate for varying wall thicknesses of the tubular conduits being joined by anastomosis.

3. The device of claim 2, wherein said second set of struts begins collapsing after said struts of said second end portion have buckled.

4. The device of claim 2, wherein said second set of struts have a higher compression strength than said struts of said second end portion.

5. The device of claim 1, wherein said is first end portion further comprises a first ring member annularly spaced about the longitudinal axis, wherein said plurality of members extending radially outwardly from said first end portion extend from said first ring member; and wherein said second end portion comprises a second ring member annularly spaced about the longitudinal axis, wherein said plurality of spaced struts extend from said second ring member toward said first ring member.

6. The device of claim 2, wherein said first end portion further comprises a first ring member annularly spaced about the longitudinal axis, wherein said plurality of members extending radially outwardly from said first end portion extend from said first ring member; and wherein said second end portion comprises a second ring member annularly spaced about the longitudinal axis; and further comprising a third ring member intermediate of said first and second ring members and annularly spaced about the longitudinal axis, wherein said plurality of spaced struts extend from said second ring member to said third ring member; and wherein said second set of spaced struts extend from said first ring member to said third ring member.

7. The device of claim 1, further comprising a plurality of tines extending radially outwardly from said second end portion.

8. The device of claim 7, wherein said plurality of tines extend from said spaced struts.

9. The device of claim 7, wherein said tines are graft tines adapted to pierce a side wall of one of the conduits to be joined by anastomosis.

10. The device of claim 1, further comprising a plurality of spaced locking tines integral with second end portion and slidably connecting with said first end portion.

11. The device of claim 10, wherein upon compression of said device, said locking tines slide with respect to said first end portion and extend beyond said first end portion, said locking tines being adapted to be bent over to lock the relative positions of said first and second end portions.

12. The device of claim 10, wherein said first end portion further comprises a plurality of eyelets axially aligned with said locking tines, through which locking tines are slidably connected to said first end portion.

13. The device of claim 1, wherein said main body defines an annular space which is configured to slide over the outer wall of one of the two conduits to be joined by anastomosis.

14. The device of claim 5, wherein said plurality of spaced struts connect said second ring member with said first ring member.

15. The device of claim 5, further comprising a plurality of tines extending radially outwardly from said second end portion and adapted to mechanically engage an everted end of a first of the tubular fluid conduits.

16. The device of claim 15, wherein said plurality of tines extend from said spaced struts.

17. The device of claim 15, wherein said plurality of tines extend from said second ring member.

18. The device of claim 1, wherein said struts are plastically deformed during buckling.

19. The device of claim 2, wherein said second set of struts are plastically deformed upon collapsing.

20. The device of claim 15, wherein the second end portion struts, upon buckling are adapted to form a compression fit with said members extending radially outwardly from said first end portion to form a seal between the everted end and an inner wall of a second of the tubular fluid conduits.

21. The device of claim 1, wherein said members extending radially outwardly from said first end portion extend substantially perpendicularly to the longitudinal axis.

22. A device for use in making an anastomosis between first and second tubular fluid conduits in the body of a patient, said device comprising a unitary structure having a main body disposed annularly about a longitudinal axis, having proximal and distal end portions, and adapted for passing the first tubular conduit through an annulus defined by the main body; an exterior of said main body configured to be inserted through an opening in the second tubular conduit; at least one member extending from said proximal end portion configured to abut against an external wall of the second conduit upon insertion of said main body through said opening; said distal end portion being configured to buckle in response to axial compression of said device, wherein said distal end portion is incapable of passing through said opening after buckling and exerts a compressive force between the first conduit and the wall of the second conduit upon buckling.

23. The device of claim 22, said distal end portion further comprising conduit retainers, wherein, upon passing the first conduit through said main body, a free end of the first conduit is everted over a distal end of the device and retained by said conduit retainers.

24. The device of claim 23, wherein said conduit retainers comprise graft tines.

25. The device of claim 23, wherein said conduit retainers are circumferentially spaced around said distal end portion.

26. The device of claim 22, wherein said distal end portion comprises buckling struts adapted to buckle in a radially outward direction upon application of a compressive force thereto.

27. The device of claim 22, wherein said at least one member extending from said proximal end portion comprises a plurality of members extending from a proximal end of said device and oriented substantially perpendicularly to the longitudinal axis.

28. The device of claim 22, further comprising locking members fixed to said distal end portion and slidably received by said proximal end portion, wherein, upon buckling of said distal end portion, free ends of said locking members slide to extend proximally of said proximal end portion.

29. The device of claim 28, wherein said proximally extending free ends of said locking members are adapted to be bent over against said proximal end portion and the external wall of the second conduit to lock the relative position of said proximal and distal end portions.

30. The device of claim 22, wherein said proximal end portion is variably collapsible to accommodate varying wall thicknesses, so as to make said device adaptable for joining the first conduit to various second conduits having varying wall thicknesses.

31. A anastomosis device comprising a unitary structure having a main body having an outside diameter adapted to pass through and form a close fit with an opening formed in a wall of a conduit to be joined by anastomosis, said main body defining an annulus having an inside diameter, said annulus extending longitudinally through said body, said body having proximal and distal end portions, an extension portion extending from said proximal end portion and configured to be incapable of passing through the opening; and conduit retainers on said distal end portion adapted to retain a free end portion of a conduit having been passed through said annulus and everted over said proximal end portion.

32. The device of claim 31, wherein said distal end portion is configured to buckle in response to axial compression of said device, and wherein said distal end portion is incapable of passing through the opening after buckling and exerts a compressive force toward said extension portion.

33. The device of claim 32, wherein said distal end portion is adapted to further evert the conduit retained by said conduit retainers upon buckling.

34. The device of claim 31, further comprising locking members fixed to said distal end portion and slidably received by said proximal end portion, wherein, upon buckling of said distal end portion, free ends of said locking members slide to extend proximally of said proximal end portion.

35. The device of claim 34, wherein said proximally extending free ends of said locking members are adapted to be bent over against said proximal end portion to lock the relative position of said proximal and distal end portions.

36. The device of claim 31, wherein said proximal end portion is variably collapsible to accommodate varying wall thicknesses that occur among different patients.

37. A device for use in making an anastomosis between first and second tubular fluid conduits in the body of a patient, said device comprising a unitary structure having a main body disposed annularly about a longitudinal axis, having proximal and distal end portions, and adapted for passing the first tubular conduit through an annulus defined by the main body; an exterior of said main body configured to be inserted through an opening in the second tubular conduit; at least one member extending from said proximal end portion configured to abut against an external wall of the second conduit upon insertion of said main body through said opening; said distal end portion being configured to buckle in response to axial compression of said device, wherein said distal end portion is incapable of passing through said opening after buckling and exerts a compressive force between the first conduit and the wall of the second conduit upon buckling; said distal end portion further comprising conduit retainers, wherein, upon passing the first conduit through said main body, a free end of the first conduit is everted over a distal end of the device and retained by said conduit retainers; and at least one locking tine integral with said distal end portion and slidably connecting with said proximal end portion.

38. A deployment instrument configured to capture an anastomosis device adapted for making an anastomosis between tubular fluid conduits in the body of a patient and comprising a unitary structure having a main body disposed annularly about a longitudinal axis, having first and second end portions and configured to be loaded with a first of the two conduits to be joined by the anastomosis, wherein the conduit is loaded by passing a free end thereof through an internal space defined by said main body in a direction from said first end portion to said second end portion and everting an end of the first conduit over said second end portion; said instrument comprising:
first and second tubes concentrically arranged for axial sliding movement with respect to one another, said first tube having a first outside diameter and further having a gradually increasing second outside diameter on a distal end portion thereof; said second tube having an inside diameter slightly greater than said first outside diameter such that said second tube is free to slide with respect to said first outside diameter of said first tube; said second tube having radially expandable members defining a radially expandable distal end portion, wherein, upon sliding said radially expandable distal end portion into contact with distal end portion of said first tube, said gradually increasing outside diameter of said first tube distal end portion drives said radially expandable members radially outward; and, upon sliding said distal end portion of said first tube out of contact with said radially expandable distal end portion of said second tube, said radially expandable members return to an unbiased, non-expanded configuration.

39. The deployment instrument of claim 38, wherein said first and second tubes each comprise a longitudinal slot, said longitudinal slots being aligned with one another and configured to allow the first conduit to pass therethrough.

40. The deployment instrument of claim 38, wherein said first and second tubes are configured to slide through the internal space defined by the main body of the device, in a direction from the first end portion to the second end portion, between an external wall of the first conduit and an internal wall of the device, when said radially expandable members are in said unbiased, non-expanded configuration.

41. The deployment instrument of claim 40, wherein said first and second tubes are configured to capture the device after sliding through the internal space, upon expanding said radially expandable members by moving said distal end portion of said first tube into contact with said radially expandable members.

42. The deployment instrument of claim 41 wherein said radially expandable members, upon radially expanding, contact and exert a force against the internal wall of the device.

43. The deployment instrument of claim 41, wherein distal ends of said radially expandable members comprise catch members that abut a distal end of the device upon said radial expansion of said radially expandable members to capture the device.

44. The deployment instrument of claim 38, further adapted to buckle the device, said instrument further comprising a stop member located proximally of said distal end portions against which said first end portion of said device abuts upon capture of said device; said first and second tubes being axially slidable in a proximal direction with respect to said stop member to exert a compressive force on said device to buckle said device.

45. The deployment instrument of claim 44, wherein said first and second tubes are axially slidable in a distal direction to release the compressive force after completion of said buckling, said first tube being adapted to further slide in an axial distal direction with respect to said second tube to remove contact between said distal end portion of said first tube and said radially expandable members; wherein said radially expandable members return to a non-expanded configuration and wherein the deployment instrument releases capture of said device.

46. The deployment instrument of claim 43, further comprising a third tube, said third tube having an inside diameter slightly greater than an outside diameter of said second tube, said third tube linked with said first tube so that said first and third tubes slide as a unit, said third tube being free to slide over said second tube when said first tube slides with respect to said second tube; said third tube having an outside radius greater than a radial extent of said catch members in a non-expanded configuration.

47. The deployment instrument of claim 46, wherein said third tube comprises a longitudinal slot aligned with said longitudinal slots of said first and second tubes and configured to allow the first conduit to pass therethrough.

48. The deployment instrument of claim 46, wherein said first, second and third tubes are configured to slide through the internal space defined by the main body of the device, in a direction from the first end portion to the second end portion, between an external wall of the first conduit and an internal wall of the device, when said radially expandable members are in said unbiased, non-expanded configuration.

49. The deployment instrument of claim 44, further adapted to lock the device after buckling the device, wherein the device has at least one locking member extending from said second end portion and slidably connected with said first end portion, and wherein upon buckling the device, a free end of each said at least one locking member slides with respect to said first end portion and extends proximally of said first end portion; said deployment instrument further comprising a device lock adapted to axially slide with respect to said first and second tubes, wherein, after compression of the device said device lock slides in a distal direction with respect to said first and second tubes, abuts said at least one free end and bends over said at least one free end of said at least one locking member, thereby locking the relative positions of the first and second end portions of the device.

50. The deployment instrument of claim 44, further comprising a force limiter interconnecting said second tube with a driving mechanism of the instrument, wherein said force limiter limits an amount of compressive force that said second tube can apply to the device.

51. The deployment instrument of claim 50, wherein said force limiter comprises an extension spring configured to begin to extend when a predetermined tensile force is applied thereto.

52. A method of performing an anastomosis to join a first conduit to a second conduit, said method comprising the steps of:
inserting a free end of the first conduit through an annular space defined by an anastomosis device comprising a unitary structure having a main body disposed annularly about a longitudinal axis and having first and second end portions; at least one first end member extending further radially outward than a radial extent of said annularly disposed main body; and graft retaining members extending from said second end portion, the graft being inserted in a direction from said first end portion to said second end portion so that the free end extends from a second end of the device;
everting the extending free end of the graft over the second end of the device and retaining the everted free end with the graft retaining members;
forming an opening through a wall of the second conduit, wherein the opening is dimensioned to allow the everted end and main body, but not the at least one first end member to pass therethrough;
inserting the device and graft into the opening until the at least one first end member abuts the external wall of the second conduit; and
compressing the device to buckle the second end portion, wherein the second end portion, upon buckling is no longer capable of passing back through the opening.

53. The method of claim 52, wherein said compressing is performed only up until a pre-defined compression force has been reached.

54. The method of claim 53, wherein said compressing further at least partially collapses said first end portion after buckling said second end portion.

55. The method of claim 52, further comprising locking the relative positions of said first and second end portions after completion of said compressing.
